(19) United States
(12) Patent Application Publication
(10) Pub. No.: US 2012/0095006 A1
(43) Pub. Date: Apr. 19, 2012

(54) TREATMENT OF PULMONARY HYPERTENSION

(75) Inventors: Ghazwan Saleem Butrous, Sandwich (GB); Timothy Michael Lukas, Sandwich (GB); Ian Machin, Sandwich (GB)

(73) Assignee: Pfizer Inc.

(21) Appl. No.: 13/328,294

(22) Filed: Dec. 16, 2011

Related U.S. Application Data
(63) Continuation of application No. 12/355,277, filed on Jan. 16, 2009, now abandoned.
Application No. 12/355,277 is a continuation of application No. 09/692,807, filed on Oct. 20, 2000, now abandoned.

(30) Foreign Application Priority Data
Nov. 2, 1999 (GB) 9925970.7
Feb. 11, 2000 (GB) 0003235.9

(51) Int. Cl. A61K 31/496; A61K 31/502; A61K 31/517; A61P 9/12; C07D 487/04; C07D 471/14; C07D 237/34; C07D 405/14; A61K 31/437; A61K 31/519

(52) U.S. Cl. 514/248; 514/252.16; 514/287; 514/254.07; 514/266.22; 514/267; 544/262; 546/64; 544/366; 544/235; 544/291; 544/251

(57) ABSTRACT

This invention relates to the use of certain cyclic guanosine 3′,5′-monophosphate phosphodiesterase type five (cGMP PDE5) inhibitors, including in particular the compound sildenafil, for the treatment of pulmonary hypertension.

This invention relates to the use of certain cyclic guanosine 3′, 5′-monophosphate phosphodiesterase type five (cGMP PDE5) inhibitors (hereinafter PDE5 inhibitors), including in particular the compound sildenafil, for the treatment of pulmonary hypertension.

According to the specification of our international patent application WO94/28902 we have discovered that compounds which are inhibitors of the cGMP PDE5 enzyme are potent and effective compounds for the treatment of male erectile dysfunction (MED, impotence) and for female sexual disorders. This discovery led to the development of the compound sildenafil (5-[2-ethoxy-5(4-methyl-1-piperazinylsulphonyl)phenyl]-1-methyl-3-n-propyl-1,6-dihydro-7H-pyrazolo[4,3-d]pyrimidin-7-one) (VIAGRA™) which has proved to be outstandingly successful as the first orally effective treatment for MED.

Pulmonary hypertension is a pathological condition in which the pulmonary arterial pressure rises above normal levels and may cause sequelae of haemodynamic changes that can become life threatening. Symptoms of pulmonary hypertension include shortness of breath with minimal exertion, fatigue, dizzy spells and fainting. When pulmonary hypertension occurs in the absence of a known cause, it is referred to as primary pulmonary hypertension. Primary pulmonary hypertension is rare, occurring in about two per million people worldwide.

Secondary hypertension is much more common occurring as a result of other medical conditions, including congestive heart failure, chronic hypoxic lung disorder, including chronic obstructive pulmonary disease, inflammatory or collagen vascular diseases such as scleroderma and systemic lupus erythematosus, congenital heart diseases associated with left to right shunting and pulmonary thromboembolism.

Since pulmonary hypertension is caused typically by constriction of the pulmonary blood vessels, vascular resistance is the favoured indicator of the disease. The pulmonary vascular resistance (PVR) and systemic vascular resistance (SVR) are calculated as follows.

$$PVR = \frac{( mean pulmonary artery pressure - pulmonary wedge pressure )}{cardiac output} \times 80$$

$$SVR = \frac{( mean arterial blood pressure - systemic venous pressure )}{cardiac output} \times 80$$

Agents which selectively lower PVR without significant lowering SVR remain limited.

The use of phosphodiesterase inhibitors administered endotracheally or endobronchially to treat pulmonary hypertension has been described in WO95/09636 but the compounds employed were neither particularly potent nor selective cGMP PDE inhibitors.

According to a first aspect, the invention provides a method of treating or preventing pulmonary hypertension in a patient which comprises treating the patient with an effective amount of a PDE5 inhibitor selected from the group:
a) sildenafil;
b) (6R, 12aR)-2,3,6,7,12,12a-hexahydro-2-methyl-6-(3,4-methylenedioxyphenyl)pyrazino[2′, 1′:6,1]pyrido[3,4-b]indole-1,4-dione;
c) 2-[2-ethoxy-5-(4-ethyl-piperazin-1-yl-1-sulphonyl)-phenyl]-5-methyl-7-propyl-3H-imidazo[5,1-f][1,2,4]triazin-4-one;

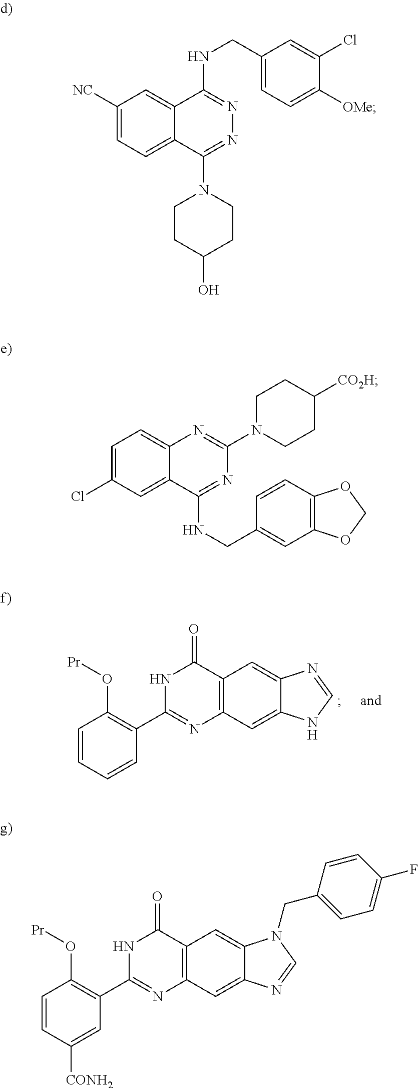

or a pharmaceutically acceptable salt, solvate or polymorph; or a pharmaceutical composition thereof.

According to a second aspect, the invention provides the use of a PDE5 inhibitor selected from the group:
a) sildenafil;
b) (6R, 12aR)-2,3,6,7,12,12a-hexahydro-2-methyl-6-(3,4-methylenedioxyphenyl)pyrazino[2′, 1′:6,1]pyrido[3,4-b]indole-1,4-dione;
c) 2-[2-ethoxy-5-(4-ethyl-piperazin-1-yl-1-sulphonyl)-phenyl]-5-methyl-7-propyl-3H-imidazo[5, 1-f][1,2,4]triazin-4-one;

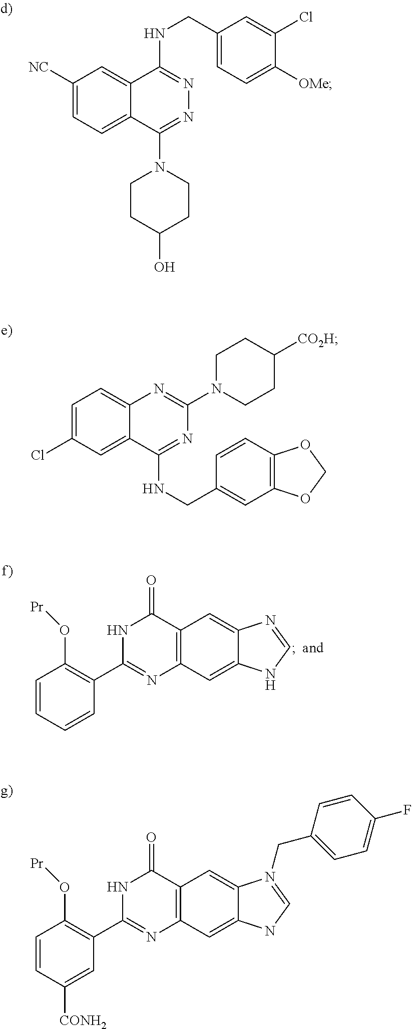

or a pharmaceutically acceptable salt, solvate or polymorph thereof; for the manufacture of a medicament for treating or preventing pulmonary hypertension.

A preferred PDE5 inhibitor is sildenafil, preferably sildenafil citrate.

For the preparation of compound a), i.e. sildenafil, (5-[2-ethoxy-5-(4-methylpiperazin-1-ylsulfonyl)phenyl]-1,6-dihydro-1-methyl-3-propylpyrazolo[4,3-d]pyrimidin-7-one) see Example 12 of EP 0463756 (incorporated herein by reference.

For the preparation of compound b), i.e. (6R, 12aR)-2,3,6,7,12,12a-hexahydro-2-methyl-6-(3,4-methylenedioxyphenyl)pyrazino[2′, 1′:6,1]pyrido[3,4-b]indole-1,4-dione (IC-351) see the compound of examples 78 and 95 of published international application WO95/19978, as well as the compound of examples 1, 3, 7 and 8 (all incorporated herein by reference).

For the preparation of compound c), i.e. 2-[2-ethoxy-5-(4-ethyl-piperazin-1-yl-1-sulphonyl) -phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5, 1-f]-as-triazin-2-yl)-4-ethoxyphenyl]sulphonyl]-4-ethylpiperazine, see compound of example 20, 19, 337 and 336 of published international application WO99/24433 (all incorporated herein by reference).

For preparation of compound d), see WO9605176 (incorporated herein by reference).

For preparation of compound e), see WO93/07124 (incorporated herein by reference).

For preparation of compounds f) and g), see Rotella D P, J. Med. Chem., 2000, 43, 1257.

Hereinafter, the PDE5 inhibitors defined in the first and second aspects are referred to as the compounds of the invention, and includes pharmaceutical salts, solvates or polymorphs thereof.

Advantageously, we have shown that compounds of the invention lower PVR to a greater extent than SVR.

### BRIEF DESCRIPTION OF THE DRAWINGS

FIG. 1 shows the effect of Sildenafil in Human Patients on PVR and SVR.

FIG. 2 shows the effect of intravenous Sildenafil in Anaesthetised Dogs on PVR and SVR.

FIG. 3 shows the effect of inhaled Sildenafil in Anaesthetised Dogs on PVR and SVR.

Compounds of the invention can be administered alone but, in human therapy will preferably be administered in admixture with a suitable pharmaceutical excipient, diluent or carrier selected with regard to the intended route of administration and standard pharmaceutical practice.

Compounds of the invention can be administered orally, bucally or sublingually in the form of tablets, capsules, ovules, elixirs, solutions or suspensions, which may contain flavouring or colouring agents, for immediate-, delayed-, modified-, or controlled-release applications. Tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose, hydroxypropylcellulose, sucrose, gelatin and acacia. Additionally, lubricating agents such as magnesium stearate, stearic acid, glyceryl behenate and talc may be included. Excipients of a similar type may also be employed as fillers in gelatin capsules. Preferred excipients in this regard include lactose, starch, a cellulose, milk sugar or high molecular weight polyethylene glycols. For aqueous suspensions and/or elixirs, compounds of the invention may be combined with various sweetening or flavouring agents, colouring matter or dyes, with emulsifying and/or suspending agents and with diluents such as water, ethanol, propylene glycol and glycerin, and combinations thereof.

In humans, oral administration of compounds of the invention is a preferred route. In circumstances where the recipient suffers from a swallowing disorder or from impairment of drug absorption after oral administration, the drug may be administered parenterally, sublingually or buccally.

Compounds of the invention can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion techniques. For such perenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Compounds of the invention can also be administered intranasally or by inhalation. Inhaled formulations have advantages in delivering the active compound directly to the lung area, producing a faster effect than orally delivered formulations. For this embodiment the aerosol particle size is preferably between 0.5 micrometers and 5 micrometers. The aerosol in conveniently generated from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide or other suitable gas. In the case of a pressurized aerosol, the dosage unit may be determined by providing a valve to deliver a metered amount. In a preferred embodiment, the compounds of the invention are administered by inhalation.

The term inhalation or inhaled includes endotracheal and endobronchial administration.

Solutions of the active ingredient for use in such inhalers are prepared by conventional methods, typically by dissolving the active ingredient in water which is preferably buffered to pH 3-8, more preferably pH 4 to 7 using standard buffer systems such as citrate, lactate or phosphate buffers to control the pH. Ethanol may also be added at a concentration up to 30% to improve aerosolisation of the formulation. Additional stabilizers may be required to improve chemical stability of the formulations; ie anti-oxidants, such as sodium metabisulphite, sodium bisulphate or tocopherol, or metal chelators such as ethylenediaminetetraacetic acid.

Single unit-dose spray can be prepared aseptically or terminally sterilized to produce a sterile final product. Alternatively, multi-dose metered nebulisers, inhalers or atomizers can be used.

Flavourings, perfumes and humectants may also be added to improve the patient acceptability of the formulation. Solubility enhancers e.g. caffeine can be added to improve solubility of the active drug.

For oral and parenteral administration to human patients, the daily dosage level of the compounds of the invention will usually be less than 500 mg (in single or divided doses). Thus, for example, tablets or capsules of sildenafil may contain less than 50 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions, compounds of the invention may be taken as a single dose on an “as required” basis (i.e. as needed or desired).

We have shown during studies on dog models, that pulmonary blood vessels are more sensitive to the actions of the compounds of the invention than is the corpus cavernosum. In the corpus cavernosum of the penis, a calculated intravenous dose of 12 microgrammes/kg sildenafil produced a half-maximal potentiation (ED50) of nerve-induced pressure rises (see Carter A J, Ballard S A and Naylor A M, The Journal of Urology, (1998), volume 160, pages 242-246). In contrast a dose of only 1.5 microgrammes/kg produced a maximal response in terms of reversing hypoxic pulmonary vasoconstriction (see Biological Studies hereinafter). This is surprising in that both effects are thought to be mediated via the inhibition of PDE5. Accordingly a preferred dose of a compound of the invention for treating pulmonary hypertension is up to 50 mg, more preferably up to 20 mg, more preferably up to 10 mg, more preferably 1 to 10 mg. Administration of such low doses to a patient significantly reduces the risk of side effects.

A preferred PDE5 inhibitor for oral administration is sildenafil (preferably sildenafil citrate), in dosages of up to 50 mg, more preferably up to 20 mg, more preferably up to 10 mg, more preferably 1 to 10 mg.

The citrate salt of sildenafil is the preferred salt for oral administration, however other pharmaceutically acceptable salts may also be used.

Alternatively the drug may be administered as a micronised powder. The drug is micronised to give a particle size in the range 0.1 to 5 micrometres, preferably less than 1 micrometre, and then blended with a suitable lactose carrier. The powder can be placed in hard gelatin capsules for use in conjunction with a conventional dry powder inhalation device.

The inhaled formulations described above should deliver a dose of a compound of the invention of up to 50 mg, more preferably up to 20 mg, more preferably up to 10 mg, more preferably 1 to 10 mg. The exact dose administered will, however, differ depending on the subject being treated, on the severity of the condition, on the manner of administration and on the judgment of the prescribing physician. Thus, because of patient-to-patient variability, the dosages given below are a guideline only and the physician may adjust doses of the compounds to achieve the treatment that the physician considers appropriate for the patient. In considering the degree of treatment desired, the physician must balance a variety of factors such as the age of the patient and the presence of other diseases or conditions (e.g. cardiovascular disease).

A preferred PDE5 inhibitor for inhaled administration is sildenafil, in dosages of up to 50 mg, more preferably up to 20 mg, more preferably up to 10 mg, more preferably 1 to 10 mg.

A preferred formulation for administration by inhalation comprises an aqueous formulation of sildenafil mesylate for use in an aerosol nebuliser or atomizer to provide a dose of less than 20 mg of sildenafil mesylate per dose.

The compounds of the invention can also be administered together with prostacyclins (e.g. Epoprostenol), oxygen, calcium channel blockers (e.g. Nifedipine, Diltazem, Amlodipine), endothelin antagonists (ETa), iloprost, adenosine and/or nitric oxide.

In addition to treatment of adult patients, a further application of the invention is in the treatment of very young children born with congenital heart disease. Compounds of the invention can be used to treat pulmonary hypertension in such subjects and can thus delay the immediate need for surgery until the patient is better able to withstand the trauma of surgery. Compounds of the invention can also be used to treat children who have pulmonary hypertension post operatively or due to respiratory distress syndrome or neonatal hypoxia.

Alternatively, compounds of the invention can be administered in the form of a suppository or pessary, or they may be applied topically in the form of a gel, hydrogel, lotion, solution, cream, ointment or dusting powder. Compounds of the invention may also be dermally or transdermally administered, for example, by the use of a skin patch. They may also be administered by the ocular, pulmonary or rectal routes.

Compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

It is to be appreciated that all references herein to treatment include curative, palliative and prophylactic treatment.

Methods of preparing such pharmaceutical compositions with a certain amount of active ingredient are well known to those skilled in this art, or may be determined by reference to literature precedents.

Examples of particular formulations are included hereinafter to further illustrate the invention. The examples are illustrative only and are not intended to limit the scope of the invention.

### EXAMPLE 1

### A Tablet Formulation for Oral Use

Sildenafil citrate (20 mg) is blended with cellulose (microcrystalline), silicon dioxide, stearic acid (fumed) and the mixture is compressed to form tablets.

### EXAMPLE 2

### An Intravenous Formulation

| | |
|---|---|
| Sildenafil citrate | 100 mg |
| isotonic saline | 1,000 ml |

### EXAMPLE 3

### Dry Powder Formulation For Inhalation

A dry powder formulation of sildenafil citrate was prepared by blending micronised drug (1 g) with lactose suitable for inhalation use, e.g. Pharmatose (trade mark), 325 mesh, (10 g) to provide a homogeneous blend. The product was filled into hard gelatin capsules (150 mg) for use with a commercial dry powder inhalation device.

Similar formulations may be prepared of sildenafil mesylate and sildenafil free base.

### EXAMPLE 4

### Solution Formulation for Inhalation

A solution was prepared of sildenafil mesylate having the following composition:

| | |
|---|---|
| Sildenafil mesylate | 10 g |
| Sodium dihydrogen phosphate | 0.69 g |
| Distilled water | 90 ml |
| Ethanol | 10 ml |

The solution was stirred to dissolve the ingredients and the pH adjusted to 4.2 by the addition of 1 M sodium hydroxide solution. The solution was filter sterilized and aseptiocally filled into amber nebuliser bottles. This solution may then be used with commercial nebulisers or atomizers to dose 10 mg of active drug.

### Preparation of 5-[2-ethoxy-5-(4-methylpiperazin-1-ylsulphonyl)phenyl]-1,6-dihydro-1-methyl -3-propylpyrazolo[4,3-d]pyrimidin-7-one) methanesulphonate salt (sildenafil mesylate)

5-[2-Ethoxy-5-(4-methylpiperazin-1-ylsulphonyl)phenyl]-1,6-dihydro-1-methyl-3-propylpyrazolo[4,3-d]pyrimidin-7-one (sildenafil, see EP 0463756 Example 12 for preparation) (100 g, 0.21 mol) was dissolved in boiling acetone (3000 ml). Methanesulphonic acid (14.9 ml, 0.23 mol) was added to the hot acetone solution. Within 10 seconds a precipitate formed. The mixture was allowed to cool and granulate for 48 hours. The title product was collected by filtration and dried in vacuum to give a white crystalline solid (116.0 g, 96.8%), m.p. 272-274° C.; Found: C, 48.33; H, 5.99; N, 14.68. C₂₃H₃₄N₆O₇S₂ requires C, 48.41; H, 6.00; N, 14.73%; δ(CD₃SOCD₃) 0.92 (3H, t), 1.33 (3H, t), 1.73 (2H, heptet), 2.29 (3H, s), 2.77 (2H, t), 2.79 (3H, s), 3.16 (2H, br), 3.3-3.57 (4H, br), 3.8 (2H, br), 4.16 (3H, s), 4.20 (2H, q), 7.4 (1H, d), 7.88 (1H, dd), 7.90 (1H, s) and 9.44 (1H, br).

### CLINICAL STUDY

The efficacy of sildenafil in pulmonary hypertension in human patients was demonstrated by the following study.

A number of patients having various causes of pulmonary hypertension were selected. The patients were then assessed in order to establish baseline data, including haemodynamic parameters (by right heart catheterization), blood gas profile (via an arterial line and pulse oximetry). The patients were then tested with 40 ppm of nitric oxide (NO) by inhilation for 5 minutes to assess the reversibility of pulmonary hypertension. Haemodynamic parameters were reassessed within 5 minutes of NO and then further assessed 5-20 minutes later. When the pulmonary arterial pressure levels had returned to baseline (+−5%) inhalation haemodynamic parameters were assessed again and patients were continuously infused with sildenafil at rates to control the plasma level at 100, 300 and 500 nanogrammes per milliliter. A few patients received placebo instead of sildenafil in double-blind fashion. Haemodynamic parameters were recorded throughout infusion.

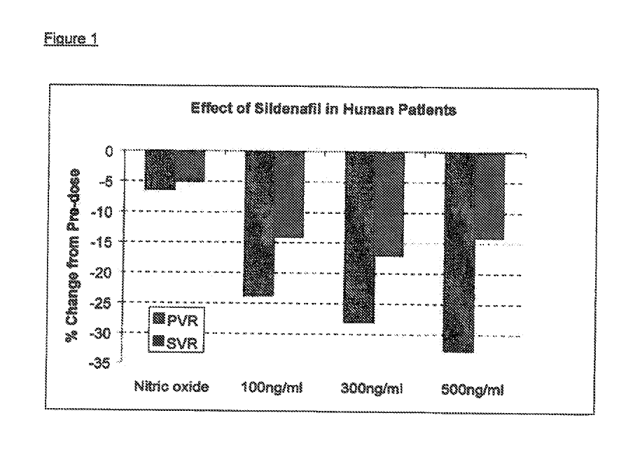

From the data collected during the trial, the PVR and SVR were determined. The results are shown in FIG. 1 and demonstrate a significant reduction in PVR experienced in a number of patients, confirming the utility of sildenafil for this indication. Furthermore, the results demonstrate that the effect of sildenafil on the SVR was substantially lower than the effect on PVR.

### BIOLOGICAL STUDIES

The efficacy of sildenafil for treating pulmonary hypertension in dogs was demonstrated by the following studies. The first study examined efficacy via intravenous administration. The second study examined efficacy via inhaled administration.

### Intravenous Administration

The anaesthetized dog is the model of choice in which to study the effect of drugs, owing to the similarity between canine and human haemodynamics, and additionally, to ensure cinsistency between pharmacological and toxicological species.

Beagle dogs of either sex were anaesthetized with intravenous sodium pentobarbitone (Sagatal [trademark], 30 to 45 mg/kg) and anesthesia maintained with an infusion of Sagatal (6 mg/kg/h) into the right femoral vein. The dogs were initially intubated via the mouth, and then via a tracheotomy, and artificially respired using an Ugo Basille dog respirator. If necessary respiratory reflexes were abolished by the administration of pancuronium (0.2 mg/kg, at 60 and 90 minute intervals). Respiratory gases (inspired O₂ and end tidal CO₂) were monitored by a Normocap 200 (trademark) respiratory gas monitor (Datex Instrumentarium Corp.). The depth of anesthesia was assessed by carefully monitoring arterial blood pressure and heart rate. The left femoral vein and artery were cannulated for dose administration and recording arterial blood pressure, respectively. The right jugular vein was cannulated with a Swan Ganz catheter, the tip of which was positioned by means of pressure monitoring in a branch of the pulmonary artery. Pulmonary artery pressure (PAP) and pulmonary artery wedge pressure were recorded from this catheter. This catheter was also connected to a cardiac index computer (Gould Instruments) and cardiac output determined by thermodilution following rapid injection of 5 ml saline at room temperature. All catheters were filled with heparinised saline. Lead II ECG was recorded from 2 stainless steel needles inserted subcutaneously in the right arm and left leg. All transducers and recording electrodes were connected to HSE preamplifiers. All data from the primary signals (PAP, Arterial blood pressure (ABP) and ECG) was recorded on a Po-ne-mah (trademark) data acquisition system. From these signals the following parameters were derived; systolic, diastolic and mean blood pressure, systolic, diastolic and mean pulmonary artery pressure, pulmonary capillary wedge pressure. Heart rate was recorded from both the blood pressure and ECG signals, for verification checks of the analysis algorithms. Arterial blood samples were taken at intervals to monitor blood gases using an ABL505 blood gas analyzer (Radiometer Ltd.). Throughout the experiment Hartmans solution was infused at a rate appropriate to maintain blood acid/base balance and volume. Core temperature was maintained at approximately 38° C. by means of a Harvard Homeothermic blanket. Following preparatory surgery, the animals were allowed to stabilize for approximately 30 minutes.

The haemodynamic parameters were logged by the data acquisition system as the mean values from complete cardiac cycles every 10 seconds. The computer file containing these values constitutes the raw data for the study. During the control periods inspired oxygen was at a concentration of 40%. Hypoxia was produced by adding nitrogen to the inspiratory gas mixture at a rate sufficient to reduce inspired oxygen to 10%. Hypoxic conditions were maintained for 15 minutes during which time cardiac output estimations were taken at 5 minute intervals. Following this hypoxic challenge the dog was returned to control conditions for 30 minutes before the next challenge. Three baseline measurements of all parameters were taken at 5 minute intervals prior to the animals receiving an hypoxic challenge. These baseline readings check the stability of the preparation. Two hypoxic challenges were conducted before the addition of any compound or vehicle.

Each intravenous dose of sildenafil consisted of a 2 minute loading infusion starting 15 minutes before an hypoxic challenge, followed by a maintenance infusion which continued throughout the hypoxic challenge and subsequent normoxia until the next dose is given. Vehicle was given at the same infusion rates as those used for sildenafil and the rate of the infusions did not exceed 2 ml/minute. A 2 ml sample of arterial blood was taken at pred dose and just prior to the end of each infusion, the plasma frozen and the drug content was analysed.

Doses were made up by dissolving sildenafil in vehicle [sodium acetate buffer (0.038 M)] to the required concentration. Four doses of 1.5, 4.5, 15 and 45 microgrammes/weight of dog in kilogrammes were administered.

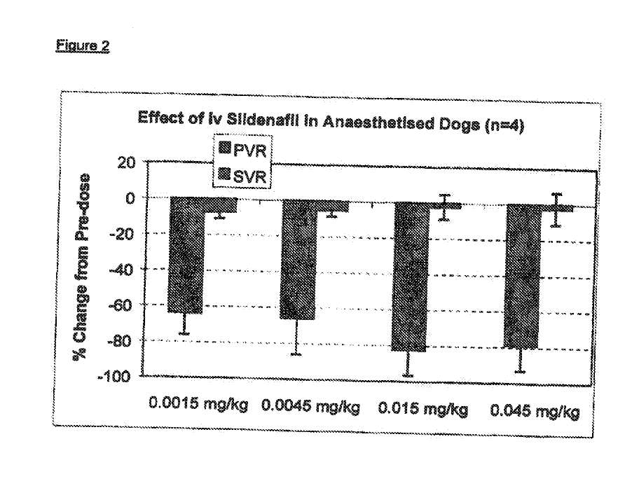

The results are shown in FIG. 2, which shows the effect on PVR and SVR against dose. The results show a maximal effect on the PVR at the lowest dose of 1.5 microgrammes/kg. The effect on SVP is significantly lower than on PVP.

### Inhaled Administration

This separate study was performed in analogous fashion to intravenous administration, except that doses were administered into the inspiratory limb of the respiratory circuit using a Penta-sonic Nebuliser (deVilbiss). Each inhaled dose consisted of a timed nebulisation of the solution at a respiration rate of 15 breaths/min finishing 5 min before an hypoxic challenge. Sildenafil and vehicle were administered to a maximum of four dogs per group (i.e. total of eight dogs). At the end of the first vehicle experiment an additional hypoxic challenge was done following administration of an inhaled dose of sildenafil.

Doses were made up by dissolving sildenafil in vehicle [sodium acetate buffer (0.038 M)] to the required concentration. Four doses of 30, 80, 230 and 770 microgrammes/weight of dog in kilogrammes were administered.

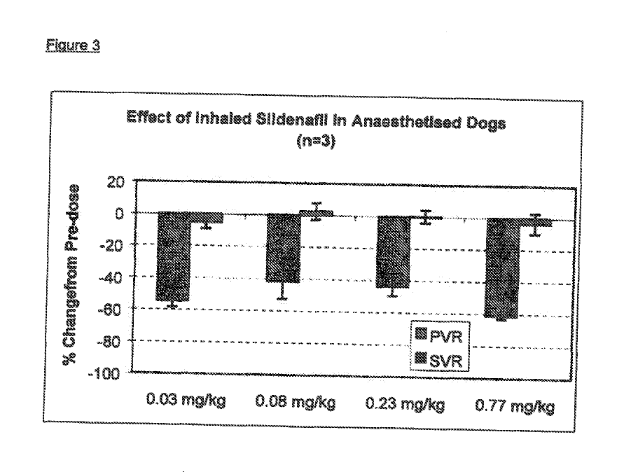

The results are shown in FIG. 3, which shows the effect of dose against PVR and SVR. The results show a maximal effect on the PVP at the lowest dose of 30 microgrammes/kg.

1. A method of treating or preventing pulmonary hypertension in a patient which comprises treating the patient with an effective amount of a PDE5 inhibitor selected from the group:
a) sildenafil;
b) (6R, 12aR)-2,3,6,7,12,12a-hexahydro-2-methyl-6-(3,4-methylenedioxyphenyl)pyrazino[2′,1′:6,1]pyrido[3,4-b]indole-1,4-dione;
c) 2-[2-ethoxy-5-(4-ethyl-piperazin-1-yl-1-sulphonyl)-phenyl]-5-methyl-7-propyl-3H-imidazo[5,1-f][1,2,4]triazin-4-one; 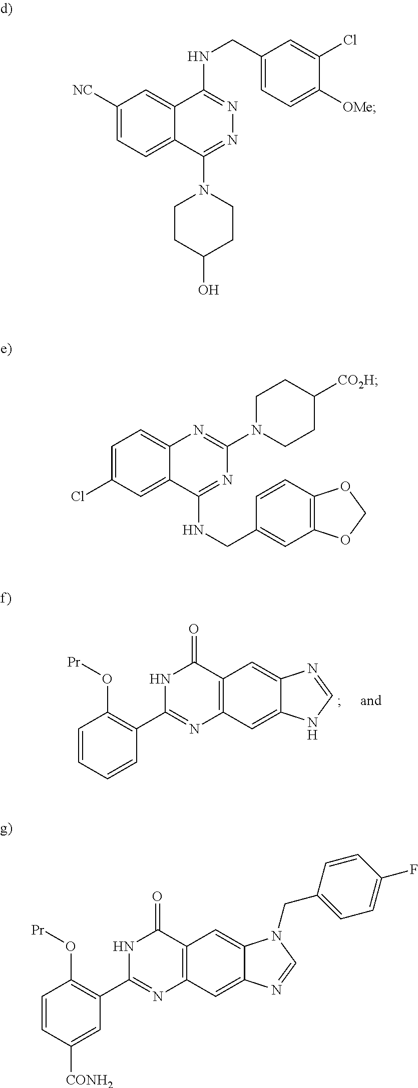
or a pharmaceutically acceptable salt, solvate or polymorph; or a pharmaceutical composition thereof.

2. A method according to claim 1 wherein the PDE5 inhibitor is sildenafil.

3. A method according to claim 1 or 2 wherein the effective amount is less than 50 mg per day.

4. A method according to claim 1 or 2 wherein the effective amount is up to 20 mg per day.

5. A method according to claim 1 or 2 wherein the effective amount is up to 10 mg per day.

6. A method according to claim 1 or 2 wherein the effective amount is from 1 to 10 mg per day.

7. A method according to any preceding claim wherein the PDE5 inhibitor is administered orally.

8. A method according to claim 7 wherein the PDE5 inhibitor is sildenafil citrate.

9. A method according to any one of claims 1 to 6 wherein the PDE5 inhibitor is inhaled.

10. A method according to claim 9 wherein the PDE5 inhibitor is sildenafil mesylate.

11. The use of a PDE5 inhibitor selected from the group:
a) sildenafil;
b) (6R, 12aR)-2,3,6,7,12,12a-hexahydro-2-methyl-6-(3,4-methylenedioxyphenyl)pyrazino[2′,1′:6,1]pyrido[3,4-b]indole-1,4-dione;
c) 2-[2-ethoxy-5-(4-ethyl-piperazin-1-yl-1-sulphonyl)-phenyl]-5-methyl-7-propyl-3H-imidazo[5,1-f][1,2,4]triazin-4-one; 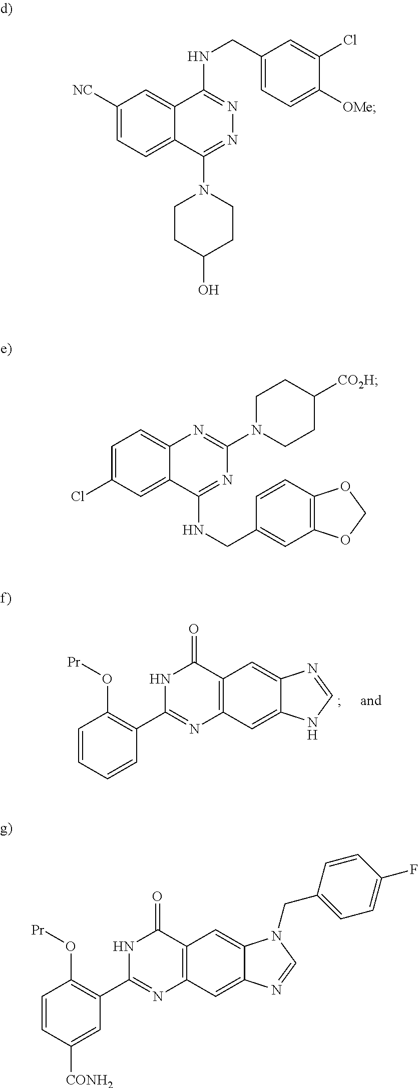
or a pharmaceutically acceptable salt, solvate or polymorph thereof; for the manufacture of a medicament for treating or preventing pulmonary hypertension.

12. The use according to claim 11 wherein the PDE5 inhibitor is sildenafil.

13. The use according to claim 11 or 12 wherein the effective amount is less than 50 mg per day.

14. The use according to claim 11 or 12 wherein the effective amount is up to 20 mg per day.

15. The use according to claim 11 or 12 wherein the effective amount is up to 10 mg per day.

16. The use according to claim 11 or 12 wherein the effective amount is from 1 to 10 mg per day.

17. The use according to any one of claims 11 to 16 wherein the PDE5 inhibitor is administered orally.

18. The use according to claim 17 wherein the PDE5 inhibitor is sildenafil citrate.

19. The use according to any one of claims 11 to 16 wherein the PDE5 inhibitor is inhaled.

20. The use according to claim 19 wherein the PDE5 inhibitor is sildenafil mesylate.
